(19) United States
(12) Patent Application Publication
(10) Pub. No.: US 2020/0360332 A1
(43) Pub. Date: Nov. 19, 2020

(54) COMPOUND FOR USE IN THE INCREASING OF MENTAL CAPACITY

(71) Applicants: LEIBNIZ-INSTITUT FUER NEUROBIOLOGIE (LIN) STIFTUNG DES OEFFENTLICHEN RECHTS, Magdeburg (DE); LEIBNIZ-INSTITUT FUER PFLANZENBIOCHEMIE (IPB) STIFTUNG DES OEFFENTLICHEN RECHTS DES LANDES SACHSEN-A, Halle (Saale) (DE); DEUTSCHES ZENTRUM FUER NEURODEGENERATIVE ERKRANKUNGEN E.V. (DZNE), Bonn (DE)

(72) Inventors: Birgit Michels, Winterhausen (DE); Bertram Gerber, Magdeburg (DE); Ludger Wessjohann, Halle (Saale) (DE); Katrin Franke, Halle (Saale) (DE); Stephan Sigrist, Berlin (DE); Anuradha Bhukel, Ashburn, VA (US); Oleh Lushchak, Ivano-Frankivsk (UA); Hanna Zwaka, Somerville, MA (US); Ruth Bartels, Berlin (DE); Alexander Dityatev, Wahlitz (DE); Inseon Song, Magdeburg (DE); Markus Fendt, Magdeburg (DE); Volkmar Lessmann, Magdeburg (DE); Thomas Endres, Magdeburg (DE); Thilo Kaehne, Magdeburg (DE)

(21) Appl. No.: 16/766,696

(22) PCT Filed: Nov. 23, 2018
(86) PCT No.: PCT/EP2018/082420

§ 371 Date: May 24, 2020

(30) Foreign Application Priority Data
Nov. 24, 2017 (DE) 10 2017 127 865.6

(51) Int. Cl. A61K 31/216; A61K 45/06; A61P 25/00

(52) U.S. Cl. CPC A61K 31/216; A61P 25/00; A61K 45/06

(57) ABSTRACT

Compound, stereoisomers, enantiomers or diastereomers thereof, physiologically acceptable salts thereof, mixtures of said compound for use in increasing mental capability, and a pharmaceutical composition. The increasing of mental capability is tested in learning experiments with Drosophila melanogaster larvae or older animals, which are raised with standard feed mash or with different concentrations of FSE-20 in the feed mash. In the learning experiment, the animals are each subjected to one of two inverse training regimens and then subjected to a test. In the training, a first scent was presented with the feed reward to the first group, while a second scent was presented alone to the first group. The second group was trained inversely. In the test of the learning experiment, it was subsequently examined whether the larvae of both groups prefer the first scent or the second scent in a selection situation; thus, their learning and memory capability was determined.

The present invention relates to a compound for use in the increasing of mental capability as well as to a pharmaceutical composition that comprises this compound.

The main risk factor for dementia is old age. Based on the demographic development of society, dementia diseases, such as, for example, Alzheimer's disease, occur ever more frequently. Parallel to this, mental fitness is playing an increasingly important role in a performance-oriented society. According to an online survey of the science journal Nature, 20% of 1400 healthy scientists resorted to pharmaceutical drugs in 2008 in order to increase their attention, concentration, or memory capability. This societal need, both in the context of dementia diseases and in the context of increased performance, has led to, among other things, the development of preparations that contain substances of synthetic or natural origin for the purpose of increasing memory capability. Preparations of plant origin are often involved.

The following compounds or plants are known in regard to learning and memory capability and are marketed in this connection: racetams (piracetam and analogs), amphetamines, methylphenidate, sabeluzole, exifone, leteprinim, AChE inhibitors (e.g., tacrine, donepezil, rivastigmine, galantamine, huperzine A (Huperzia serrata (Thunb.) Trevis), omega-3 fatty acids (e.g., fish oil, Salmo salar L.), curcumin (Curcuma longa L.), vincamine (Vinca minor L.), Ginkgo biloba L., Panax ginseng C. A. Mey, Bacopa monnieri (L.) Wettst., Evolvulus alsinoides L., Lepidium meyenii Walp., Eleutherococcus senticosus (Rupr, & Maxim.) Maxim, Erythroxylum coca Lam, Zingiber officinale Roscoe, Hypericum perforatum L., and Rhodiola rosea L.

The exact effects of these substances, particularly of many plant products, on memory are usually not known. The products found on the market are predominantly composed of plant extracts, which have been standardized to individual constituents of the plant parts used (reference substance), for which, however, no causal relationship whatsoever need be connected to the effect. Therefore, the bioactive substance within the plant part is often not in accord with the reference substance, since the reference substance is usually chosen in accordance with analytical aspects—for example, as typical components of the plant in question—in order to avoid confusion with similar plants. In such cases, strongly varying concentrations of the bioactive substance within different product batches are to be anticipated and result in variations in the effectiveness of the product and incorrect doses. This fundamental problem in the use of plant-based preparations can be solved only by the use of specific active substances having a causally demonstrated activity.

The object of the present invention is therefore to overcome these drawbacks of the prior art and to provide a compound that, in comparison to the known substances, can specifically and significantly increase mental capability.

The object is achieved by providing a compound for use in the increasing of mental capability in accordance with the features of the main claim. Advantageous embodiments of the compound according to the invention are characterized in the dependent subclaims.

The subject of the present invention is a compound having the general formula I,

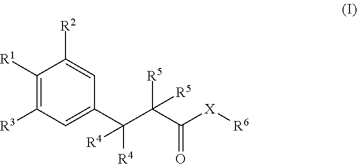

wherein

R¹, R², and R³ are chosen independently of one another from the group consisting of H, hydroxy, methoxy, ethoxy, n-propoxy, and i-propoxy;

R⁴ and R⁵, each independently of the other, are H or, in each case, an R⁴ and an

R⁵ together form a C—C bond in such a way that, between the mutually neighboring C atoms at which R⁴ and R⁵ are located, there is a single, double, or triple bond;

R⁶ is chosen from the group consisting of unbranched or branched C₆ to C₆₀ alkyl, unbranched or branched C₆ to C₆₀ alkenyl, C₆ to C₆₀ hydroxypolyethylene glycol, C₆ to C₆₀ methoxypolyethylene glycol, and C₅ to C₁₂₀ prenyl; and

X is chosen from the group consisting of O, S, N—H, N-methyl, N-ethyl, N-n-propyl, N-i-propyl, and N—R⁶;

the stereoisomers, enantiomers, or diastereomers thereof and the physiologically acceptable salts thereof as well as mixtures of the cited compounds for use in the increasing of mental capability.

Preferred is a compound for use in the increasing of mental capability having the general formula I, wherein R¹ and R² are, independently of each other, hydroxy and/or methoxy.

Particularly preferred is a compound for use in the increasing of mental capability wherein R¹ is hydroxy and R² is methoxy.

Further preferred is a compound for use in the increasing of mental capability wherein R³ is H.

Preferred, moreover, is a compound for use in the increasing of mental capability wherein the C—C bond formed by R⁴ and R⁵ is a double bond.

Particularly preferred is a compound for use in the increasing of mental capability wherein the double bond is an E double bond.

Especially preferred is a compound for use in the increasing of mental capability wherein X is O.

Moreover, a compound for use in the increasing of mental capability is preferred wherein R⁶ is a C₈ to C₂₈ alkyl, preferably a C₁₂ to C₂₄ alkyl and, more preferably, a C₈ alkyl, a C₁₂ alkyl, a C₁₆ alkyl, or a C₂₀ alkyl.

Furthermore, a compound for use in the increasing of mental capability is preferred wherein the compound is chosen from the group consisting of

icosyl (2E)-3-(4-hydroxy-3-methoxyphenyl)prop-2-enoate,

octyl (2E)-3-(4-hydroxy-3-methoxyphenyl)prop-2-enoate,

hexadecyl (2E)-3-(4-hydroxy-3-methoxyphenyl)prop-2-enoate,

icosyl 3-(4-hydroxy-3-methoxyphenyl)propanoate,

dodecyl (2E)-3-(4-hydroxy-3-methoxyphenyl)prop-2-enoate, and

icosyl (2E)-3-(4-hydroxyphenyl)prop-2-enoate.

Especially preferred is a compound for use in the increasing of mental capability wherein the mental capability comprises memory capability and the learning capability and is associated with a dementia disease.

Preferred, moreover, is a pharmaceutical composition for use in the increasing of mental capability that comprises at least one compound according to the invention as well as the physiologically acceptable salts and stereoisomers, enantiomers, diastereomers, or mixtures thereof and pharmaceutically acceptable excipients and additives. In the sense of the present invention, the pharmaceutical composition may also comprise combinations of a plurality of the compounds according to the invention.

Especially preferred is a pharmaceutical composition for use in the increasing of mental capability in combination with additional active substances, which are chosen from the group consisting of nutritional supplements, synergistic enhancers, additive enhancers, compounds for the improvement of absorption and transport, and agents for the reduction of cleavage or metabolism as well as mixtures thereof.

Particularly preferred is a pharmaceutical composition for use in the increasing of mental capability wherein the pharmaceutical composition is compounded for oral, intranasal, or subcutaneous administration.

In the sense of the present invention, the mental capability comprises memory capability as well as learning capability. Memory capability is understood to mean the ability of the brain to receive information, to store it, and to retrieve it at a later point in time. The information that is to be stored can be diverse in nature. It can involve sensations and experiences, as well as learning material, numbers, names, facts, and current issues.

The term learning describes the process of acquiring or changing behavioral patterns or cognitive structures. Learning cannot be observed directly, but rather only the learning capability, which is manifested during or after a learning operation. Learning is spoken of when a subject, as a result of more or less passively produced experience and/or one's own activity (and the consequences thereof) change or changes one's behavior in a specific situation (in response to a specific stimulus) more than just temporarily.

Furthermore, in the sense of the present invention, mental capability is also associated with humans and animals that suffer from a dementia disease. Dementia is the general term for disease symptoms that entail a loss of mental capability, such as thinking, remembering, orienting, and linking thought content, that is, a loss of memory and learning capability, and leads to the loss of any ability to perform everyday activities independently. Included, among other things, are Alzheimer's disease, vascular dementia, Pick's disease, and frontotemporal dementia.

In the sense of the present invention, the increasing of mental capability is also understood to mean an improvement of mental capability or else the restoration of mental capability.

In the sense of the present invention, nutritional supplements can comprise amino acids as well as performance-enhancing nutritional supplements, such as caffeine.

In the sense of the present invention, synergistic or additive enhancers can be caffeine, nicotine, or one of the above-mentioned known products for increasing memory.

Furthermore, in the sense of the present invention, the compounds for the improvement of absorption and transport can be chosen from the group consisting of liposomes, cyclodextrins, lysine, polyethylene glycol, cholic acid, and phytosterols.

In the sense of the present invention, agents for the reduction of cleavage or metabolism can be ester hydrolase inhibitors, inhibitors of efflux from cells, and inhibitors of metabolism in the liver, such as, for example, inhibitors of oxidizing enzymes.

The present invention will be explained in detail by way of the appended figures and drawings. Shown are:

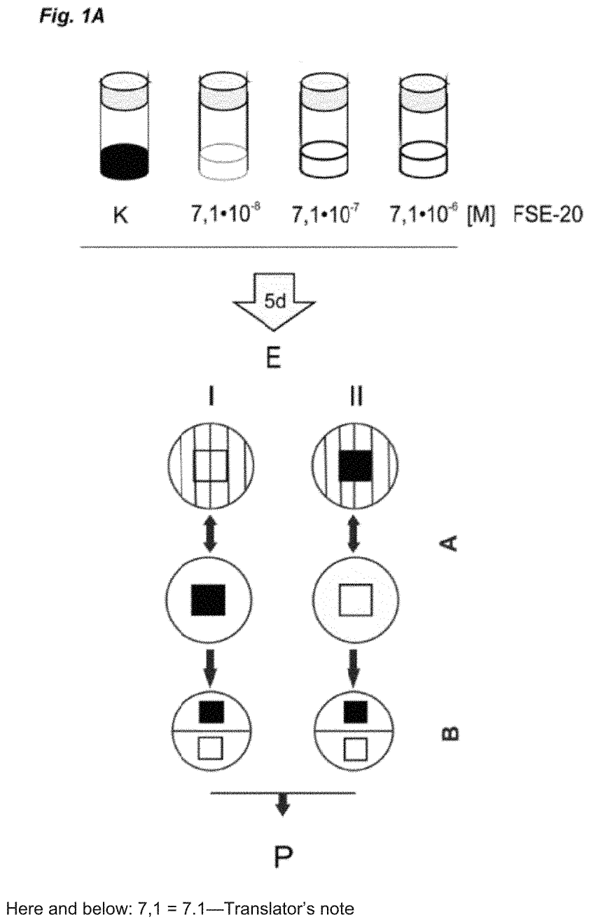

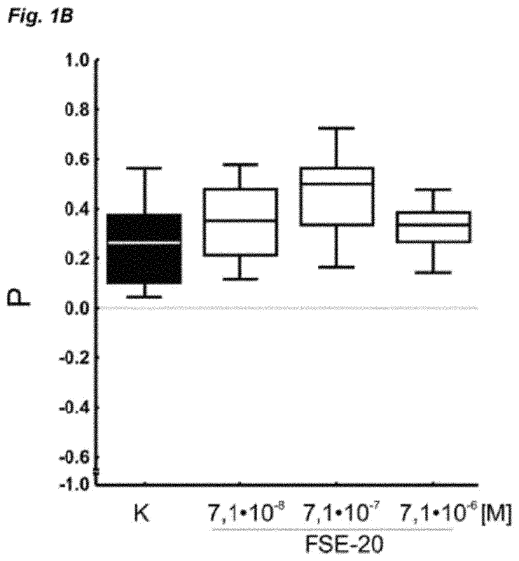

FIG. 1A: an overview of a learning experiment after feeding eicosanyl ferulate to Drosophila melanogaster larvae;

FIG. 1B: a graphic illustration that depicts the results of the learning experiment in accordance with FIG. 1A;

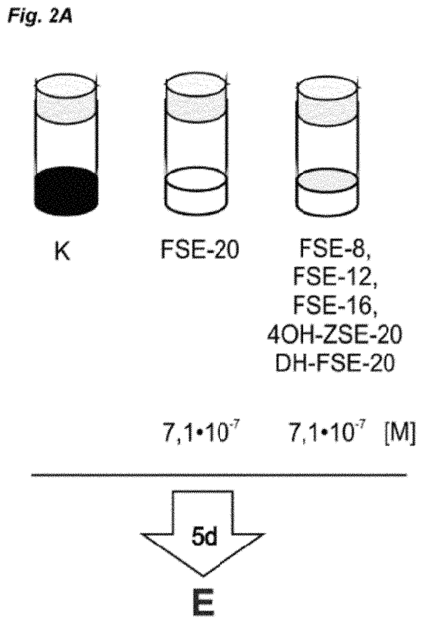

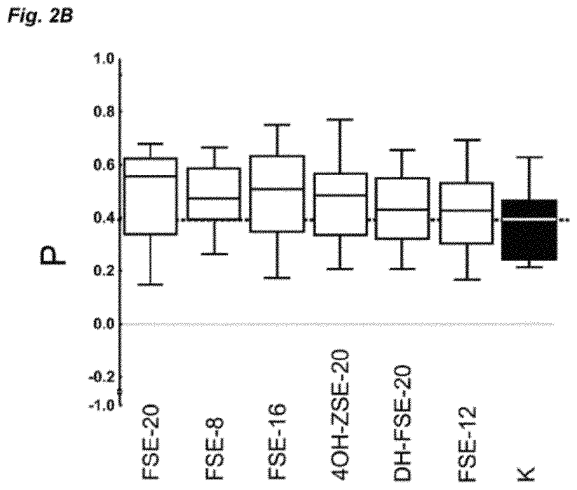

FIG. 2A; an overview of a learning experiment after feeding eicosanyl ferulate or derivatives thereof to Drosophila melanogaster larvae;

FIG. 2B: a graphic illustration that depicts the results of the learning experiment in accordance with FIG. 2A;

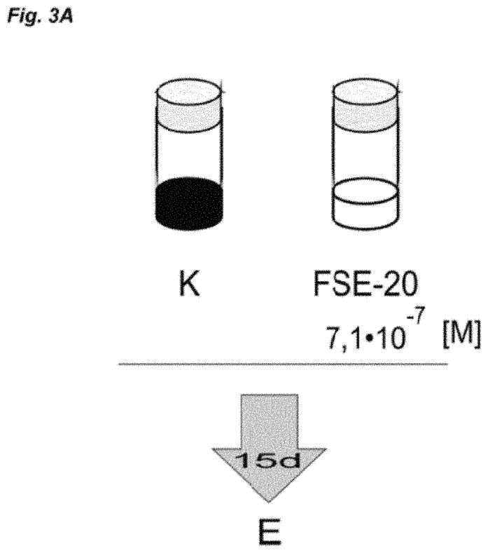

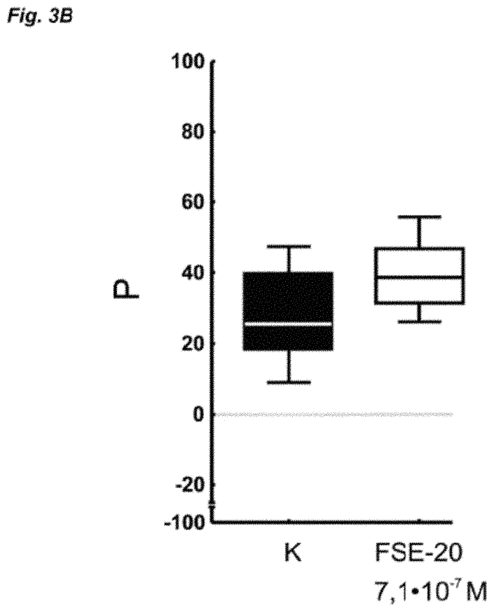

FIG. 3A: an overview of a learning experiment after feeding eicosanyl ferulate to older Drosophila melanogaster flies;

FIG. 3B: a graphic illustration that depicts the results of the learning experiment in accordance with FIG. 3A;

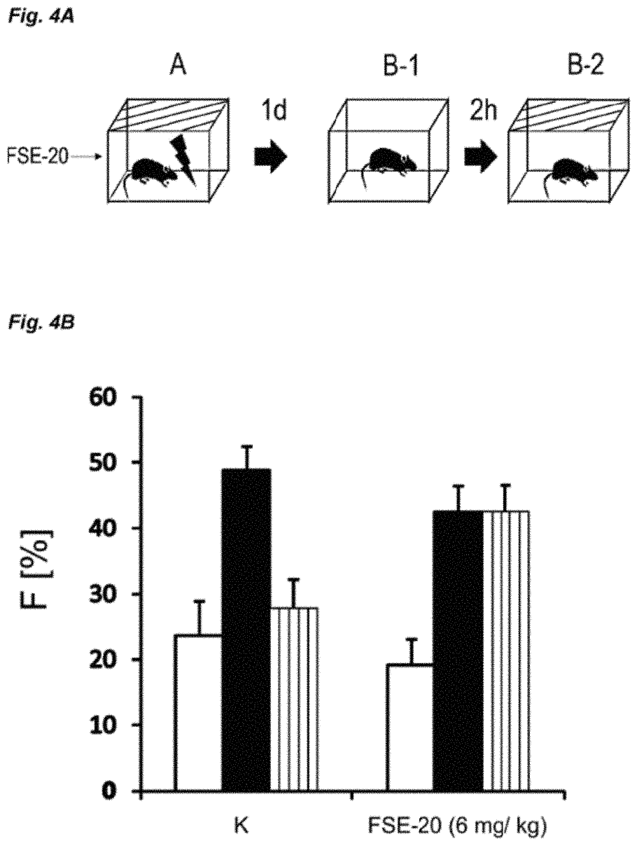

FIG. 4A: an overview of a learning experiment in mice after injection of eicosanyl ferulate;

FIG. 4B: a graphic illustration that depicts the results of the learning experiment in accordance with FIG. 4A;

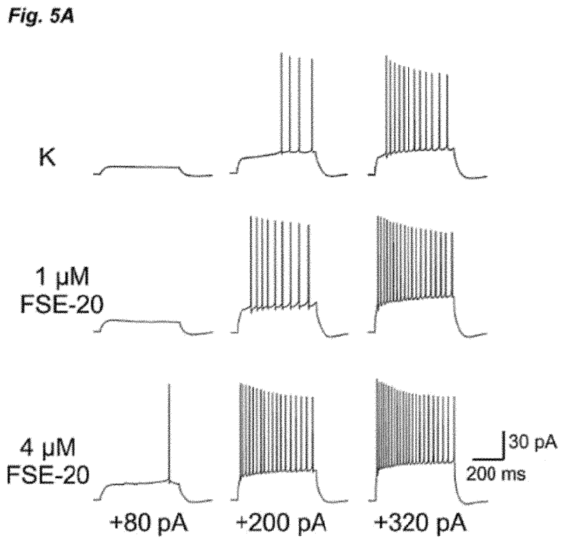

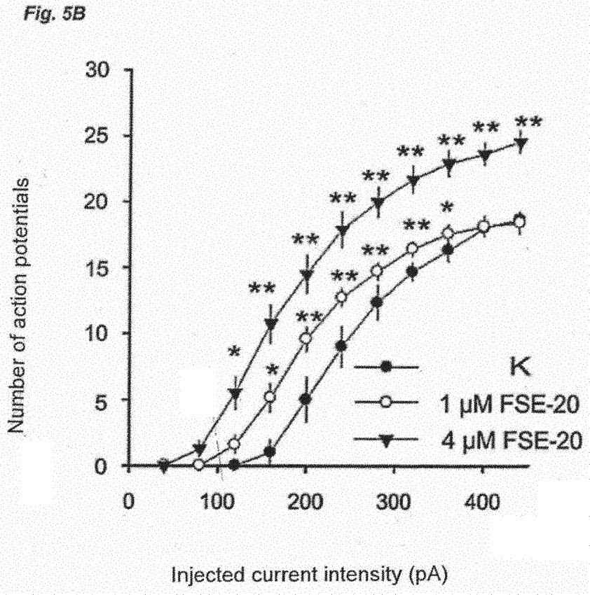

FIG. 5A: Illustrations of ex vivo electrophysiological recordings on hippocampal CA1 mouse cells after they have been treated with eicosanyl ferulate;

FIG. 5B: a graphic illustration that depicts the number of action potentials of CA1 pyramidal cells in accordance with FIG. 5A in relation to the injected current intensity.

The compounds having the general formula I are lipophilic esters of cinnamic acid and derivatives thereof, in particular derivatives of ferulic acid. Esters of cinnamic acid are referred to below as “ZSE” and esters of ferulic acid are referred to as “FSE.”

In comparison to the known substances of the prior art, these compounds have the advantage that they can increase specifically and significantly the mental capability in humans and animals. Accordingly, it is also possible to increase attention and concentration. Of further advantage is the fact that an exact dose is possible and thus any variation in effectiveness can be excluded.

Thus, it is of further advantage that these compounds can also be used for the treatment of dementia, in particular Alzheimer's disease. Dementia is characterized, in particular, in that it entails a loss of mental capability. Accordingly, the increasing of mental capability through use of compounds according to the present invention would also be advantageous for the treatment of dementia.

The pharmaceutical composition of the present invention is prepared in a suitable dose in a known way by using the excipients and additives that are conventionally used in pharmaceutical technology depending on the desired type of application.

Preferably, the pharmaceutical composition is adjusted for oral, intranasal, or subcutaneous administration.

Forms of administration of the pharmaceutical composition are, for example, tablets, lozenges, caplets, dragées, capsules, pills, powders, solutions, aerosols, suspensions, depot forms, or parenteral preparations, such as solutions for injection. Preferred for oral application, in particular, are forms of administration that can be given as food and/or feed, drinks, or nutritional supplements.

The production of the pharmaceutical preparations according to the invention is known as such and is described in the handbooks known to the person skilled in the art, such as, for example, Hager's Handbuch (5th edition) 2, 622-1045; List et al., Arzneiformenlehre, Stuttgart: Wiss. Verlag 1985; Sucher et al. Pharmaceutical Technologie, Stuttgart: Thieme 1991; Ullmann's Technologie, Berlin: Ullstein Mosby 1995.

Surprisingly, the inventors have found that the use of a compound according to formula I in Drosophila melanogaster as well as in mice leads to a significant increasing of mental learning capabilities.

The addition of eicosanyl ferulate (FSE-20) to the feed of larvae of the fruit fly Drosophila melanogaster increases their learning and memory capability significantly in a dose-dependent manner. Furthermore, it was also found that the learning capability of older flies is doubled. Other synthesized FSEs likewise lead to increases in learning in Drosophila larvae, although the improvement in learning capability is less pronounced when there is a significant shortening of the alkyl chain of the alcohol component of the ester (effect of FSE-20>FSE-16>>FSE-8) or when the double bond is hydrogenated to form 2,3-dihydroferulic acid (DH-FSE-20). Long-chain esters of para-coumaric acid (4-hydroxycinnamic acid (4OH-ZSE-20)), which lack the methoxy group in comparison to ferulic esters, are characterized by a weaker, but still marked activity. Furthermore, the inventors have found that the most active compounds have an effect even in the nanomolar range. For example, FSE-20 has an effect at 700 nM in feed mash.

Furthermore, the inventors have found that, in mice, the injection of FSE-20 prior to the learning experiment leads to a memory that is more stable over time. Moreover, ex vivo experiments show that the excitability of pyramidal CA1 hippocampus cells increases in a dose-dependent manner in mice treated with FSE-20. It is known that the pyramidal CA1 hippocampus cells, both in rodents and in humans, are involved in learning capabilities and memory capabilities.

Because the surprisingly found effects are based on the administration of a single substance, in contrast to the application of a plant extract, an exact dose and the avoidance of overdoses of the bioactive substance are ensured.

An extension to humans is indicated because the neuromolecular mechanisms of learning and of memory are similar to a high degree in humans and animals, including the fruit fly used here. In addition, mice that have been injected with FSE-20 have a more stable memory and ex vivo experiments on hippocampal CA1 cells show an increase in the excitability of these cells when they have been treated with an FSE-20 solution. Because a decrease in cellular excitability is associated with the decline in cognitive abilities with age and in the case of dementia, this also indicates that FSE-20 promotes some forms of memory by increasing the cell excitability

In accordance with the present invention, the following compounds are therefore especially preferred forms of implementation:

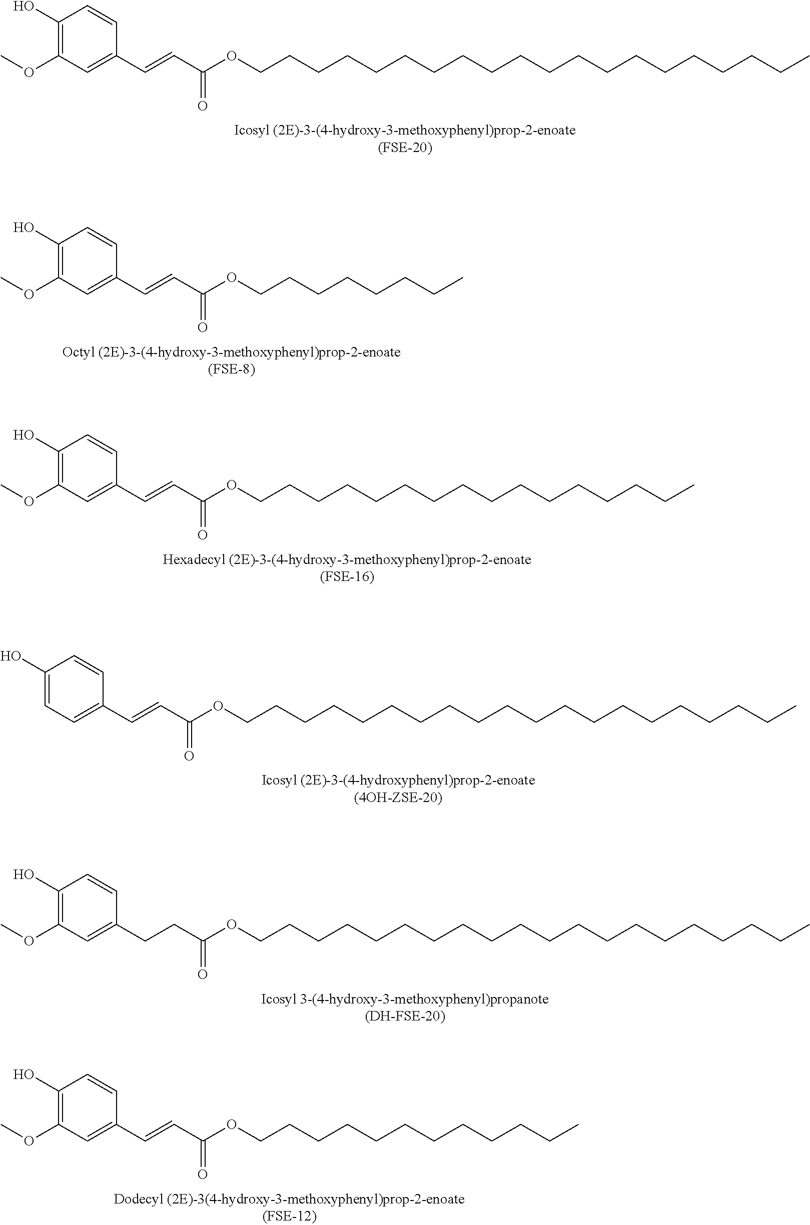

The present invention will be presented once more below in other wording:

The subject of the present invention relates to the use of esters of general formula IA

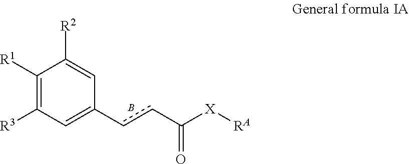

with

B=single bond, double bond (E or Z), or triple bond, wherein an E double bond is preferred;

X=O, S, NH, N-Me, N-Et, N—Pr. N—R^{A}; wherein O is preferred;

R^{A}=lipophilic chain, preferably C₆-C₆₀ alkyl, C₆-C₆₀ alkenyl, C₆-C₆₀ polyalkenyl, or C₆-C₆₀ isoalkyl, C₆-C₆₀ PEG-OH or C₆-C₆₀ PEG-OMe, oligoprenyl (C₅-C₁₂₀), and, more preferably, C₁₂-C₂₈ alkyl; R¹, R²; R³=H, OH, OMe, OEt, OPr in any combination, preferably with R¹, R²=OH and/or OMe in any combination and R³=H;

wherein ferulic acid derivatives (R¹=OH, R²=OCH₃, R³=H) are most especially preferred,

and/or combinations (mixtures) of the above compounds for the increasing of general mental capability in humans and animals, preferably for the increasing of learning capability and/or memory capability in humans and animals. Further preferred is the use of esters of the general formula IA for the treatment of dementia diseases, in particular Alzheimer's disease.

Further preferred is the use of ferulic esters of long-chained unbranched and/or simply methyl-branched alcohols (R^{A}), also in any mixtures. Preferably used in this case, in particular, are the naturally occurring long-chain alkyl esters of cinnamic acid, in particular, the tetracosanol, docosanol, heneicosanol, eicosanol, nonadecanol, octadecanol, hexadecanol, and dodecanol esters of ferulic acid. The use of esters of ferulic acid with R^{A}=C₁₆-C₂₄ is preferred, most particularly eicosanyl ferulate (C₂₀ ester).

Further preferred is the use of the aforementioned compounds in food and/or feed, including drinks and/or nutritional supplements and/or pharmaceutical preparations of any kind.

The use of the aforementioned substances and preparations in the form of prodrugs, in particular those in which a phenolic group is protected by a temporally proximate or biotically cleavable group—for example, as a phenolic ester, especially the acetate or succinate of ferulic acid—is additionally of advantage.

Further preferred is the use of the aforementioned compounds also in combination with other active substances, in particular performance-enhancing substances (e.g., caffeine) and/or nutritional supplements, including amino acids, and/or matrices and/or synergistic enhancers and/or preparations for the improvement of absorption or transport (e.g., liposomes, cyclodextrins, lysine, PEGs, cholic acid, and phytosterols or other lipophilic carriers) and/or agents or encapsulations for reducing cleavage and metabolism. However, this list merely presents preferred implementations and is not intended to impose any limitation on the active substances, agents, and preparations mentioned.

The use of the aforementioned substances and preparations is preferred especially for non-oral application, particularly for use in nasal sprays or for use in products for transdermal application.

The following examples explain the invention in more detail without limiting the scope of the invention.

### EXAMPLE 1

Increasing the memory capability of Drosophila melanogaster larvae by feeding of synthesized FSE-20.

Depicted in FIG. 1A is an overview of a learning experiment E with Drosophila melanogaster larvae. The overview shows that the larvae were divided into four groups. One group represents the control group K, which was raised with standard feed mash, and the three other groups were each raised with different concentrations of FSE-20 (7.1×10⁻⁸, 7.1×10⁻⁷, and 7.1×10⁻⁶[M] FSE-20) in the feed mash. After 5 days (arrow with “5d”), the learning experiment E was carried out.

In the learning experiment E, the animals were each subjected to one of two inverse training regimens A and then subjected to a test B. The first group I was presented in training A with a first scent with the feed reward (fructose), while a second scent was presented alone (first scent+/second scent). In FIG. 1A, the first scent is depicted as a white square and the second scent as a black square. Furthermore, a Petri dish containing fructose is represented by a white circle with longitudinal stripes and a Petri dish that does not contain fructose is represented by a white circle without the longitudinal stripes. The second group II was trained inversely (first scent/second scent+). In test B of the learning experiment E, it was subsequently examined whether the larvae of the two groups I, II prefer the first scent or the second scent in a selection situation and thus their learning and memory capability (performance index) P was determined.

Shown in FIG. 1B is a graphic illustration that depicts the results of the learning and memory capability P tested in the learning experiment E. Accordingly, the learning and memory capability P of larvae that are grown either with standard feed mash (control K, black box) or with 7.1×10⁻⁸, 7.1×10⁻⁷, or 7.1×10⁻⁶ M FSE-20 solution (white boxes) in the feed mash are compared. This graphic illustration reveals that the learning and memory capability P of all larvae groups that have been grown with FSE-20 solutions in the feed mash is increased in comparison to the control group K. Furthermore, the graphic illustration reveals that, in the case of very low concentrations (70 nM), an increase in learning tendency is manifested. At 700 nM, the learning and memory capability P has even improved substantially by approximately 50%, although, at very high concentrations starting at 7 μM it declines once again.

### EXAMPLE 2

Comparison of the learning and memory capability P of Drosophila melanogaster larvae after feeding synthesized FSE-20 or derivatives thereof.

Shown in FIG. 2A is an overview with the compounds used in this example as additions to the feed mash. One group of the Drosophila melanogaster larvae was the control group K, which was raised with standard feed mash. Furthermore, one group was raised with FSE-20 in a concentration of 7.1×10⁻⁷ M in the feed mash. The other groups were raised with a concentration of 7.1×10⁻⁷ M of the different FSE-20 derivatives in the feed mash. These FSE-20 derivatives comprise FSE-8, FSE-12, FSE-16, 4OH-ZSE-20, and DH-FSE-20. After five days (arrow with “5d”), the Drosophila melanogaster larvae were tested in the learning experiment E, as described in Example 1.

Shown in FIG. 2B is a graphic illustration that summarizes the following results of the learning experiment E:

The control group K shows no increase in the learning and memory capability P. In contrast, the addition of FSE-20 to the feed mash brings about a marked increase in the learning and memory capability P in the larvae.

The derivatives of FSE-20 also increase the learning and memory capability P, as summarized below.

It was shown that FSE-8 increases the learning and memory capability P of Drosophila larvae. To this end, the substance was admixed to the feed mash with a final concentration of 700 nM. Furthermore, it was shown that, at a final concentration of 700 nM in the feed mash, FSE-16 also acts on the animals in a manner that increases learning. The addition of DH-FSE-20 in a final concentration of 700 nM to the feed mash also has an effect that increases learning and memory in the larvae.

The addition of 4OH-ZSE-20 to the feed mash of the Drosophila melanogaster larvae, at a concentration of 700 nM, shows a slight increase in the learning and memory capability P of the animals. The addition of FSE-12 in a final concentration of 700 nM to the feed mash also brings about only a slight increase in the learning and memory capability P in the animals.

### EXAMPLE 3

Increasing the learning and memory capability P of mature, older Drosophila melanogaster flies by means of synthesized FSE-20.

FIG. 3A shows an overview of the conduct of the learning experiment E with older flies. In this experiment, the flies were divided into two groups, a control group K, which was fed with standard feed, and a test group, for which FSE-20 was added to the feed. The concentration of FSE-20in the feed was 7.1×10⁻⁷ M. The flies were tested in a learning experiment E, corresponding to the principle of Example 1, 15 days (arrow with “15d”) after the adult animals had hatched.

FIG. 3B shows a graphic illustration that depicts the results of the learning experiment E. It shows that the learning and memory capability P in older animals that had FSE-20 in the fly feed improved substantially by up to approximately 50%.

### EXAMPLE 4

Learning experiment E with mice after injection of FSE-20.

FIG. 4A shows an overview of the learning experiment E in mice. In order to determine the effect of FSE-20 on the learning and memory capability P in 3- to 4-month-old mice, the animals underwent intraperitoneal injection with an FSE-20 solution (6 mg/kg body weight) prior to the training A. In the learning experiment E on the next day, the animals were placed in a specific environment during the training A, while the mice were subjected at the same time to an aversive electrical stimulus (lightning symbol). Subsequently, in the first test B-1 one day later (1 d), the nonspecific fear reaction (freezing) F was measured in a neutral environment, that is, in an environment that was new for the animals. In the second test B-2, two hours (2 h) after the first test B-1, the fear reaction in the environment was measured, in that the animals were presented with the electrical stimulus during the training A (conditioned environment).

Shown in FIG. 4B is a graphic illustration that summarizes the results of the learning experiment E on mice. Depicted is the fraction of the time during which the two mice groups (control K and group treated with FSE-20) showed a fear reaction (freezing) F, which, during the first test B-1 in the neutral environment, that is, new environment, for the animals (white bars) and then in the second test B-2, was measured in two successively following spans of time within the conditioned environment. The first span of time of the second test comprises the 1st-5th minute and the second span of time comprises the 6th-10th minute. The result of the first span of time is represented as black bars and the result of the second span of time is represented as white bars with black longitudinal stripes. For mice treated with FSE-20, a fear behavior F that is just as strongly pronounced is shown in the second half of the learning experiment E (6th-10th minute) as in the first half (1st-5th minute), whereas, in the second half, the control K shows a strongly reduced fear behavior F. In consequence of this, memory loss or extinction has already set in for the control group at this point in time, whereas the animals treated with FSE-20 have a more stable memory over time.

### EXAMPLE 5

Ex vivo electrophysiological recordings on hippocampal CA1 mouse cells with FSE-20.

The patch-clamp method was carried out on whole CA1 pyramidal cells from “acute” (freshly prepared) hippocampus slices of four-to-six-week-old male mice in order to measure the effects of FSE-20 on neuronal excitability.

FIG. 5A shows illustrations of the representative reaction of action potentials of the CA1 cells to three successive electric stimulations of +80, +200, and +320 pA. Shown are the action potentials of the control K as well as those of the cells treated with FSE-20. The middle illustration shows the action potentials of the cells treated with 1 μM FSE-20 and the bottommost illustration shows the action potentials of the cells treated with 4 μM FSE-20.

Depicted in FIG. 5B is a graphic illustration in which the number of action potentials is shown in relation to the injected current intensity (pA) of the differently treated cells and of the control group K.

The number of action potentials that are generated in response to current intensities of 40 to 440 pA is increased significantly in the two groups treated with FSE-20 (1 μM and 4 μM) in comparison to the control K. Accordingly, the higher cell excitability in the presence of FSE-20 indicates that FSE-20 promotes some forms of memory, because, in contrast, a decrease in the cellular excitability is associated with the decline in cognitive abilities with age and in the case of dementia.

### LIST OF REFERENCE CHARACTERS

A training

B test

E learning experiment

F fear reaction

K control or control group

P learning and memory capability (performance index)

I first group

II second group

1. A compound according to the general formula I 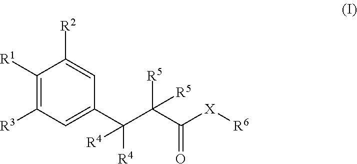
wherein
R¹, R², and R³ are chosen independently of one another from the group consisting of H, hydroxy, methoxy, ethoxy, n-propoxy, and i-propoxy;
R⁴ and R⁵, each independently of the other, are H or, in each case, an R⁴ and an
R⁵ together form a C—C bond in such a way that, between the mutually neighboring C atoms at which R⁴ and R⁵ are located, there is a single, double, or triple bond;
R⁶ is chosen from the group consisting of unbranched or branched C₆ to C₆₀ alkyl, unbranched or branched C₆ to C₆₀ alkenyl, C₆ to C₆₀ hydroxypolyethylene glycol, C₆ to C₆₀ methoxypolyethylene glycol, and C₅ to c₁₂₀ prenyl; and
X is chosen from the group consisting of O, S, N—H, N-methyl, N-ethyl, N-n-propyl, N-i-propyl, and N—R⁶;
the stereoisomers, enantiomers, or diastereomers thereof and the physiologically acceptable salts thereof as well as mixtures of the cited compounds for use in the increasing of mental capability.

2. The compound for use in the increasing of mental capability according to claim 1, further characterized in that R¹ and R² are, independently of each other, hydroxy and/or methoxy.

3. The compound for use in the increasing of mental capability according to claim 2, further characterized in that R¹ is hydroxy and R² is methoxy.

4. The compound for use in the increasing of mental capability according to claim 2, further characterized in that R³ is H.

5. The compound for use in the increasing of mental capability according to claim 1, further characterized in that the C—C bond formed by R⁴ and R⁵ is a double bond.

6. The compound for use in the increasing of mental capability according to claim 5, further characterized in that the double bond is an E double bond.

7. The compound for use in the increasing of mental capability according to claim 1, further characterized in that X is O.

8. The compound for use in the increasing of mental capability according to claim 1, further characterized in that R⁶ is a C₈ to C₂₈ alkyl, preferably a C₁₂ to C₂₄ alkyl, and, more preferably, a C₈ alkyl, a C₁₂ alkyl, a C₁₆ alkyl, or a C₂₀ alkyl.

9. The compound for use in the increasing of mental capability according to claim 1,
further characterized in that the compound is chosen from the group consisting of icosyl (2E)-3-(4-hydroxy-3-methoxyphenyl)prop-2-enoate,
octyl (2E)-3-(4-hydroxy-3-methoxyphenyl)prop-2-enoate,
hexadecyl (2E)-3-(4-hydroxy-3-methoxyphenyl)prop-2-enoate,
icosyl 3-(4-hydroxy-3-methoxyphenyl)propanoate,
dodecyl (2E)-3-(4-hydroxy-3-methoxyphenyl)prop-2-enoate, and
icosyl (2E)-3-(4-hydroxyphenyl)prop-2-enoate.

10. The compound for use in the increasing of mental capability according to claim 1, wherein the mental capability comprises memory capability and learning capability and is associated with a dementia disease.

11. A pharmaceutical composition for use in the increasing of mental capability, comprising at least one compound according to claim 1 as well as the physiologically acceptable salts and stereoisomers, enantiomers, diastereomers, or mixtures thereof, and pharmaceutically acceptable excipients and additives.

12. The pharmaceutical composition for use in the increasing of mental capability according to claim 11, in combination with additional active substances, which are chosen from the group consisting of nutritional supplements, synergistic enhancers, additive enhancers, compounds for improving absorption and transport, and agents for the reduction of cleavage or metabolism, as well as mixtures thereof.

13. The pharmaceutical composition for use in the increasing of mental capability according to claim 11, further characterized in that the pharmaceutical composition is prepared for oral, intranasal, or subcutaneous administration.
